(19) United States
(12) Patent Application Publication
(10) Pub. No.: US 2025/0195468 A1
(43) Pub. Date: Jun. 19, 2025

(54) METHODS AND COMPOSITIONS FOR TREATING COLORECTAL CANCER WITH INDOXYL SULFATE

(71) Applicant: National Institute of Immunology, New Delhi (IN)

(72) Inventors: Nishu Dalal, New Delhi (IN); Anil Kumar, New Delhi (IN)

(21) Appl. No.: 18/539,826

(22) Filed: Dec. 14, 2023

(51) Int. Cl. A61K 31/404; A61K 45/06; A61P 35/00

(52) U.S. Cl. CPC A61K 31/404; A61K 45/06; A61P 35/00

(57) ABSTRACT

In the present invention, the role of indoxyl sulfate (IS) has been investigated for its selective anti-cancer activity on colon cancer cells. IS treatment to HCT-116 and HT-29 human epithelial adenocarcinoma cells led to a decrease in cell proliferation, cell viability and ATP content. Colon cancer cells showed a 10% increase in cell apoptosis in comparison to a control. Due to IS treatment, cell morphology was distorted, cell number found decreased, intracellular vesicles formed, and cells were found floated in the media. Cells also showed loss in membrane integrity and decrease in colony forming ability and ceased at G2/M phase of cell cycle. No significant change was noted in the level of inflammatory cytokines IL-17A, IL-1β and TNF-α, histology, length of intestine and spleen, after 100 mM IS treatment to balb/c mice. These observations indicate selective anticancer effect of IS to colon cancer cells.

### FIELD

The present invention relates to the field of cancer therapy, in particular, colorectal cancer. The present invention further relates to use of Indoxyl Sulfate as anticancer agent, pharmaceutical composition and method of treatment of cancer using Indoxyl Sulfate.

### BACKGROUND

Colorectal cancer is the third most frequent diagnosed cancer in the world. It is anticipated that the CRC burden will enhance with 1.6 million deaths and 3.2 million fresh cases by 2040, with most of these expected in countries with high or very high HIDs. With growing rates in countries in transition and among young adults, there is an urgent need to better understand the outcomes and act accordingly to prevent the disease from causing future cases and deaths. In recent years, research endeavours for devising the treatment strategies for combating the colorectal cancer are being focused on the role of intestinal microbial metabolites in inhibiting and advancing cancer. Intestinal bacterial metabolites namely short-chain fatty acids, indoles derivatives, bacteriocins. and phenyl propanoid-derived metabolites exhibited direct and indirect anticancer effect by various molecular processes.

Gut microbiota produces indole compounds by degrading aromatic amino acid i.e. tryptophan which further get transformed into Indoxyl Sulfate (IS) with the help of liver cytochrome oxidases. There are a number of reports about anti-cancer activity of indole derivatives and some of them such as Vinblastine and Sunitinib have been used in clinic evaluations and cancer therapy.

Among gut microbial metabolites, indole and indole-derivatives are generated from tryptophan by particular microbial species. While indoles compounds are implicated in the communication of the gut microbial community, these molecules are also active on the mucous membrane of the intestine, usually having positive outcome in a variety of experimental situations. After absorption, indole is partly metabolised in the liver into indoxylsulphate.

Indole is a multi-purpose pharmacophore and a remarkable heterocyclic compound with a broad range of pharmacological properties inclusively anticancer activities. Indole derivatives could carry out anticancer activity by many different mechanisms like influencing apoptosis (Mcl-1 inhibitors/myeloid cell leukemia-1), replication and transcription (DNA topoisomerases), epigenetic modifications (histone acetyltransferases/HATs inhibitors, histone deacetylase/HDAC inhibitors and silent mating type information regulation 2 homolog/SIRT inhibitors), the signal transduction in cells (proviral insertion site in moloney murine leukemia virus/Pim inhibitors) and cell mitosis. Moreover, many indole-containing compounds like Nintedanib, Sunitinib, Semaxanib, Vincristine, Vinblastine, and Vinorelbine have been used previously in medical exercise for the cure of a variety of cancers, including drug-resistant cancer. Hence, the indole fraction is an effective pharmacophore for the generation of new anticancer drugs that may be active against various cancers, also for drug-resistant forms.

Indoles are absorbed by intestine and then go to liver by means of blood. In liver, consecutive hydroxylation and sulfation of these by products further converts them into indoxyl sulfate by hepatic cytochrome oxidases CYP2E1 and SULTIAI respectively which then come into blood again.

Recently, a study found that indoxyl sulfate has cytotoxic effect on breast cancer cells. According to the study, cytotoxic features were relied on increased oxidative stress that blocked the ability of cancerous cells to translocate, enter blood vessels and metastasize. In the same study, indoxyl sulfate reduces the amount of cancer stem cells which are extremely resistant to chemotherapy and play an essential function in triggering relapse. Indoxyl sulfate shows its role via the PXR and AHR receptors, and AHR appears as more dominant receptor. Indolepropionic acid, a further metabolite of tryptophan that has cytostatic effects on breast cancer, its effects on both receptors are more balanced. Lower PXR and AHR expression associated with enhanced disease grade, stage, and increased mitotic activity within the tumor. Administration of indoxyl sulfate lowers the infiltration into surrounding tissues, cell proliferation, and metastasizing in cell and animal models. This characteristic is shared by other cytostatic breast cancer metabolites.

Based on the previous reports about anti-cancer activity of indole and indole derivatives, the Applicant has investigated the potential of the indoxyl sulfate for its anti-cancer activity on HCT-116 and HT-29 human epithelial adenocarcinoma cell lines.

The inventors have carried out different cell assays such as viability assay, apopotic assay, cell cycle assay. LDH assay and more to determine if IS has any cytostatic activity on colon cancer cells.

Moreover, the effect of IS on normal colonic cells by using balb/c mice was investigated to check if IS administration is safe and does not cause any inflammation or harm to colonic cells of animal.

Finally, it was determined that IS has deleterious effect on cancer cells but is safe for normal colonic cells, hence can be proposed as anti-cancer agent for colorectal cancer treatment.

### SUMMARY

In the present disclosure, the role of indoxyl sulfate, an indole derivative, for its anti-cancer activity on colon cancer cells has been investigated. Further, it has been examined as to whether indoxyl sulfate has selective deleterious effects on cancer cells and causes no harm to normal colonic cells.

The HCT-116 and HT-29 human epithelial adenocarcinoma cell lines were taken to study the deleterious effect of IS on cancer cells. The cells were treated with 0.018 mM to 10 mM concentrations of indoxyl sulfate for three incubation periods i.e., 24 hrs, 48 hrs and 72 hrs. After IS treatment, decrease in cell proliferation efficiency, cell viability and ATP content was observed. Colon cancer cells showed 10% increase in cell apoptosis in comparison to control.

Due to treatment, cell morphology got disturbed, cell number was found to decrease, cells got distorted, intracellular vesicles formed and cells were found floating in media. Cells also showed loss in membrane integrity and decrease in colony forming ability.

Indoxyl sulfate also caused G2/M cell cycle to cease.

During animal study, balb/c mice were treated with 100 mM of indoxyl sulfate to check colonic inflammation, if any, by IS administration and level of inflammatory cytokines IL-17A, IL-1β and TNF-α were studied. The change in the level of inflammatory cytokines after IS treatment was found statistically insignificant, hence it was inferred that IS is not causing inflammation.

Histological study also showed no sign of inflammation after IS treatment. No significant change in the length of intestine and spleen and body weight of animals was noted after IS treatment.

In conclusion, it was found that IS has selective deleterious effect on colon cancer cells and does not cause harm to normal colonic cells, hence IS can be considered as potential anti-cancer agent, warranting further investigation in this direction.

The following presents a simplified summary of the different aspects of the invention. The summary is not to be construed as an extensive overview of the disclosure and it does not identify key/critical elements or delineate the scope of the invention. The sole purpose of the below summary is to present few concepts of the invention in a simplified form as a prelude to the more detailed description that is presented in detailed description of the invention.

The present invention relates to an anticancer agent comprising Indoxyl Sulfate for treatment of colorectal cancer.

In another aspect, the anticancer agent comprises Indoxyl Sulfate at a concentration range of 0.038 mM to 10 mM.

In yet another aspect, the anticancer agent comprises Indoxyl Sulfate present at a concentration of 10 mM.

In yet another aspect, the anticancer agent comprises Indoxyl Sulfate present at a concentration of 5 mM.

In yet another aspect, the present invention relates to a pharmaceutical composition for inhibiting activity of colon cancer cells comprising a therapeutically effective amount of anticancer agent and a pharmaceutically acceptable excipient.

In another aspect, the pharmaceutical composition comprises anticancer agent comprising of Indoxyl Sulfate (IS) at a concentration range of 0.038 mM to 10 mM.

In another aspect, the pharmaceutical composition comprises anticancer agent comprising of Indoxyl Sulfate (IS) at a concentration of 10 mM.

In yet another aspect, the pharmaceutical composition comprises anticancer agent comprising of Indoxyl Sulfate (IS) at a concentration of 5 mM.

In another aspect, the present invention relates to a method for treating or preventing colorectal cancer in a subject, said method comprising administering to the subject in need thereof a therapeutically effective of Indoxyl Sulfate and any mixture containing Indoxyl Sulfate.

In yet another aspect, Indoxyl Sulfate is administered alone, in a pharmaceutical composition or in combination with other cancer therapy in the said method. The other cancer therapy comprises surgery, chemotherapy, immunotherapy or radiation therapy.

In another aspect, the method involves a subject being a mammal.

In yet another aspect, the invention relates to use of Indoxyl Sulfate as an anticancer agent for treatment of colorectal cancer.

### BRIEF DESCRIPTION OF THE DRAWINGS

The foregoing summary, as well as the following detailed description of the invention will be better understood when read in conjunction with the appended drawings. For the purpose of assisting in the explanation of the invention, there are shown in the drawings embodiments which are presently preferred and considered illustrative. It should be understood, however, that the invention is not limited to the precise arrangements and instrumentalities shown therein.

FIGS. 1A-1B. Effects of indoxyl sulfate on cell viability by MTT assay. The HCT-116 (A) and HT-29 (B) cells were treated with varying levels of IS over 24 hours, 48 hours, and 72 hours. Cell viability was ascertained using MTT assay. Untreated cells were taken as controls and determined to be 100% viable. Different mean values across controls and treated cells were analysed by ANOVA. Cells show decrease in cell viability in concentration dependent manner while proved most cytotoxic at 10 mM IS treatment for 72 hours.

FIGS. 2A-2B. Effects of indoxyl sulfate on cell viability by CellTiter-Glo luminescent cell viability assay. The HCT-116 (A) and HT-29 (B) cells were exposed with varying levels of IS over 24 hours, 48 hours, and 72 hours. Cell viability have been evaluated by utilizing CellTiter-GloR Assay kit. Luminescence was recorded and taken as directly proportional to amount of ATP. The luminescence of non-treated cells was perceived as control and considered 100% viable. Different mean values across controls and treated cells were analysed by ANOVA. Cells show decrease in cell viability in concentration dependent manner while proved most cytotoxic at 10 mM IS treatment for 72 hours.

FIGS. 3A-3B. Effects of indoxyl sulfate on membrane integrity by LDH assay. After treatment of HCT-116 (A) and HT-29 (B) cells with different concentration of IS for 48 hours, LDH release was assessed in supernatant of HCT-116 and HT-29 cells. Absorbance value of positive control was considered as 100%. Different mean values across controls and treated cells were analysed by ANOVA. Cells show significant decrease in membrane integrity in concentration dependent manner while proved most cytotoxic at 10 mM IS.

FIGS. 4A-4B. Detection of apoptosis by Annexin V-FITC/PI double staining-flow cytometry dot plot. Impact of IS on HCT116 (A) and HT29 (B) cells assessed through flow cytometry, after treatment with different concentration of IS for 48 hours. Dot plot showing the percentages of no. of necrotic cells (annexin V-FITC−, PI+), viable cells (annexin V-FITC−, PI−), early apoptotic cells (annexin V-FITC+, PI−) and late apoptotic cells (annexin V-FITC+. PI+). Results shows a significant increases in late apoptosis with increase in IS concentration.

FIGS. 5A-5B. Detection of apoptosis by Annexin V-FITC/PI double staining-graphical representation. After exposing the HCT-116 (A) and HT-29 (B) cells with various concentration of IS for 48 hours, apoptosis was measured in HCT-116 and HT-29 cells by flow cyotometry as represented in histogram. Different mean values across controls and treated cells were analysed by ANOVA. Cells show significant increase in late apoptosis in concentration dependent manner while proved most cytotoxic at 10 mM IS.

FIGS. 6A-6B. Effects of indoxyl sulfate on colony formation by clonogenic assay. After treatment of HCT-116 (A) and HT-29 (B) cells with distinct concentration of IS for 48 hours, no. of colonies was counted by using ImageJ software as represented in histogram. Different mean values across controls and treated cells were analysed by ANOVA. Cells show significant in colony formation in concentration dependent manner while proved most cytotoxic at 10 mM IS.

FIGS. 7A-7B. Effect of indoxyl sulfate on cell cycle progression-flow cytometry histogram. After treatment of HCT-116 (A) and HT-29 (B) cells with distinct concentration of IS for 48 hours, cell distribution in various phases of cell cycle was quantified by flow cytometry. Distribution of cells are increasing with concentration in G2/M phase and causing G2/M cell cycle arrest. IS induce cell cycle arrest in concentration dependent manner.

FIGS. 8A-8B. Effect of indoxyl sulfate on cell cycle progression-graphical representation. After treatment of HCT-116 (A) and HT-29 (B) cells with various concentration of IS for 48 hours, cell distribution in various phases of cell cycle was measured in cells by flow cytometry as portrayed in histogram. Different mean values across controls and treated cells were analysed by ANOVA. IS induce cell cycle arrest in concentration dependent manner while proved most cytotoxic at 10 mM IS.

FIGS. 9A-9B. Effects of indoxyl sulfate on intestinal and spleen length. After sacrificing the mice, the intestinal (A) and spleen (B) length (cm) was measured which shows that there is no significant change after treatment with IS as compared to vehicle and negative control groups.

FIGS. 10A-10B. Effects of indoxyl sulfate on colon and rectum histology Histology of colon and rectum was checked at the end of 12th week by staining with hematoxylin and eosin and images were taken in light microscope at 40×. Images shows that there is no change in histology of mice as compared to vehicle and negative control groups after treatment with indoxyl sulfate.

FIGS. 11A-11C. Effects of indoxyl sulfate on inflammatory cytokines levels. IL-17 (A), IL-1β (B) and TNF-α (C) level was checked at zero, 1 month and 2 month of treatment with IS by intraperitoneal (IP) and intrarectal (IR) administration. Different mean values across controls and treated cells were analysed by ANOVA. There is no significant change in IL-17, IL-1β (B) and TNF-α level after treatment with IS as compared to vehicle and negative control groups.

FIGS. 12A-12B. Effect of indoxyl sulfate on cell morphology. HCT-116 (A) and HT-29 (B) cells were exposed with various concentration of IS over 24 hrs, 48 hrs and 72 hrs. Cell morphology was checked by bright field microscopy with a 20× objective. Bright field images showed changes in cell morphology, decrease in cell numbers and detachment of cell from culture substrate with increased IS concentration and incubation time.

FIGS. 13A-13B. Effect of indoxy lsulfate on colony formation by clonogenic assay. After treatment of HCT-116 (A) and HT-29 (B) cells with various concentration of IS for 48 hrs, cells were evaluated for their colony formation ability. Result show that IS inhibits the colony formation ability in concentration dependent fashion.

FIG. 13. Effect of indoxyl sulfate on intestinal and spleen length. After sacrificing the mice, the intestinal and spleen length (cm) was measured. No significant change was observed after intraperitoneal (IP) and intrarectal (IR) IS treatment as compare to vehicle and negative control groups. Standard error between six mice taken in one group is represented with error bars.

### DETAILED DESCRIPTION

In describing the invention, the following terminology will be used in accordance with the definitions set forth below. Unless defined otherwise, all technical and scientific terms used herein have the same meaning as commonly understood by one of ordinary skill in the art to which this invention belongs.

Although any methods and materials similar or equivalent to those described herein can be used in the practice or testing of the present invention, the preferred methods and materials are described herein.

As used herein, each of the following terms has the meaning associated with it in this section. Specific and preferred values listed below for individual process parameters, substituents, and ranges are for illustration only: they do not exclude other defined values or other values falling within the preferred defined ranges.

As used herein, the singular forms “a.” “an,” and “the” include plural reference unless the context clearly dictates otherwise.

As used herein, the terms “comprising” “including,” “having.” “containing,” “involving.” and the like are to be understood to be open-ended, i.e. to mean including but not limited to.

Although any methods and materials similar or equivalent to those described herein can be used in the practice or testing of the present invention, the preferred methods and materials are described. Numeric ranges are inclusive of the numbers defining the range.

The use of examples anywhere in this specification including examples of any terms discussed herein is illustrative only, and in no way limits the scope and meaning of the invention or of any exemplified term. Likewise, the invention is not limited to various embodiments given in this specification.

The detailed description of the present invention is not limited in its application merely to the exemplifications of construction and the arrangement of components set forth in the following description. The present disclosure is capable of encompassing other embodiments and of being practiced or of being carried out in various ways.

Also, the phraseology and terminology used herein is for the purpose of description and should not be regarded as limiting.

The present invention relates to an anticancer agent comprising Indoxyl Sulfate for treatment of colorectal cancer.

In another aspect, the anticancer agent comprises Indoxyl Sulfate at a concentration range of 0.038 mM to 10 mM.

In yet another aspect, the anticancer agent comprises Indoxyl Sulfate present at a concentration of 10 mM.

In yet another aspect, the anticancer agent comprises Indoxyl Sulfate present at a concentration of 5 mM.

In yet another aspect, the present invention relates to a pharmaceutical composition for inhibiting activity of colon cancer cells comprising a therapeutically effective amount of anticancer agent and a pharmaceutically acceptable excipient.

In another aspect, the pharmaceutical composition comprises anticancer agent comprising of Indoxyl Sulfate (IS) at a concentration range of 0.038 mM to 10 mM.

In another aspect, the pharmaceutical composition comprises anticancer agent comprising of Indoxyl Sulfate (IS) at a concentration of 10 mM.

In yet another aspect, the pharmaceutical composition comprises anticancer agent comprising of Indoxyl Sulfate (IS) at a concentration of 5 mM.

In another aspect, the present invention relates to a method for treating or preventing colorectal cancer in a subject, said method comprising administering to the subject in need thereof a therapeutically effective of Indoxyl Sulfate and any mixture containing Indoxyl Sulfate.

In yet another aspect, Indoxyl Sulfate is administered alone, in a pharmaceutical composition or in combination with other cancer therapy in the method for treating or preventing colorectal cancer.

In a preferred embodiment of the invention, the other cancer therapy comprises surgery, chemotherapy, immunotherapy or radiation therapy.

In yet another aspect, the cancer is locally advanced or a metastatic cancer.

In one more embodiment of the invention, the said subject is diagnosed with Stage 0, Stage I, Stage II, Stage III or Stage IV cancer.

In another aspect of the invention, Indoxyl Sulfate is administered in a pharmaceutical preparation, a nutritional preparation or a food preparation.

A further aspect of the invention discloses that the Indoxyl Sulfate is administered in a dosage amount from about 100 mg/Kg body weight to 125 mg/Kg body weight of subject under treatment.

In yet another aspect, the method involves a subject being a mammal. In one more aspect of the invention, the present invention provides use of Indoxyl Sulfate as an anticancer agent for treatment of colorectal cancer.

### Materials and Methods Used in the Invention.

### Chemicals

Chemicals, kits and reagents were purchased from Sigma-Aldrich and Dextran sodium sulfate (DSS) (cat. no. 160110) was purchased from MP Biomedicals. The purity of all the chemicals is >95% by HPLC analysis.

### Cell Culture

Human colon epithelial adenocarcinoma cell lines, HT-29 and HCT-116 (procured from ATCC), are commonly utilized for CRC research. HT-29 cells were cultured in McCoy 5A media (Gibco) supplemented with 10% heat-inactivated FBS (Gibco) and penicillin-streptomycin (Sigma-Aldrich: Merck KGaA, Germany). On the other hand, HCT-116 cells were cultured in Dulbecco's modified Eagle's medium-F12 nutrient mixture (DMEM-F12: Gibco) supplemented with 10% heat-inactivated FBS (Gibco) and penicillin-streptomycin (Sigma-Aldrich: Merck KGaA, Germany). The cells were incubated in a humidified incubator at 37° C. with 5% CO2.

To investigate cell proliferation, the cells were treated with indoxyl sulfate at concentrations of 1.25 mM, 5 mM, and 10 mM for incubation periods of 24 hours, 48 hours, and 72 hours. After the specified treatment durations, the cells were harvested using 0.1% trypsin-EDTA. All experiments were conducted during the exponential growth phase.

### Cell Viability

To evaluate the impact of IS treatment, cell viability was assessed using Thiazolyl Blue Tetrazolium Bromide (MTT) dye. HT-29 and HCT-116 cells were seeded at a density of 3000 cells per well in a 96-well plate. After 24 hours of seeding, the cells were exposed to various concentrations of IS, including 0.018 mM, 0.037 mM, 0.075 mM, 0.15 mM. 0.31 mM, 0.62 mM. 1.25 mM, 2.5 mM, 5 mM, and 10 mM. Triplicate wells were treated with IS for three different incubation periods: 24 hours, 48 hours, and 72 hours. After the respective incubation periods. 20 μl of MTT dye (5 mg/ml) was added to each well and incubated for 3 hours. The yellow MTT dye is converted into purple formazan crystals by the mitochondrial succinate dehydrogenase enzyme, NAD (P) H. Following the incubation, 100 μl of dimethyl sulphoxide (DMSO) was added to dissolve the formazan crystals. Formazan crystal dissolve immediately after adding DMSO, so taken the reading just after adding the DMSO. The intensity of the purple color is directly proportional to cell viability. Absorbance was measured at 570 nm using a Multiskan Sky microplate spectrophotometer (Thermo Scientific). Non-treated cells served as controls and were considered 100% viable.

### Luciferase Assay

Cell viability was also assessed using the CellTiter-Glo R: Assay kit, which measures the amount of ATP to indicate cellular metabolic activity. HT-29 and HCT-116 cells were cultured in a 96-well plate at a density of 2500 cells per well. After 24 hours of seeding, the cells were exposed to various concentrations of IS, including 0.018 mM, 0.037 mM, 0.075 mM. 0.15 mM, 0.31 mM, 0.62 mM, 1.25 mM, 2.5 mM, 5 mM, and 10 mM. Triplicate wells were treated with IS for two different incubation periods: 48 hours and 72 hours. After the respective incubation periods. the assay was performed following the manufacturer's protocol. The details of the protocol can be found in the kit provided by the manufacturer. In summary, luminescence was measured using a Sirius Luminometer V3.2 (Berthold DETECTIONN SYSTEMS) with a measurement delay time of 2 seconds and a measurement time of 5 seconds. Non-treated cells were used as controls, representing 100% metabolically active cells in terms of ATP content.

### LDH Assay

Membrane integrity was assessed using the LDH based in vitro toxicology kit obtained from Sigma-Aldrich. The assay was conducted following the manufacturer's protocol. HCT-116 and HT-29 cells were cultured in a 96-well plate at a density of 10,000 cells per well. After 24 hours of seeding, the cells were treated with IS at concentrations of 1.25 mM, 5 mM, and 10 mM. Following a 48-hour treatment period, the cells were centrifuged, and 100 μl of supernatant was collected into a fresh 96-well plate to measure the amount of LDH released into the media by necrotic cells. Subsequently, 100 μl of the dosing reagent was added and incubated for 30 minutes at 37° C. After incubation, 20 μl of IN HCl stop solution was added. Absorbance was measured at 690 nm and 490 nm using a ClarioStar plate reader (BMG Labtech, Ortenberg, Germany). The background absorbance at 690 nm was subtracted from the 490 nm absorbance. For the positive control, 0.1% Triton X-100 was used and considered as maximum LDH release. Untreated cells served as the negative control. The membrane integrity of IS-exposed cells was compared to that of untreated cells. The following formula was used to calculate the LDH release from the cells: Experimental LDH release (OD490)×100/maximum LDH release (OD490).

### Apoptosis Assay

The apoptotic effect of IS was evaluated on HCT-116 and HT-29 cells using the propidium iodide (PI)/Annexin V-fluorescein isothiocyanate (FITC) dual staining kit (ab14085: Abcam, USA). Duplicate wells of cells were cultured at a density of 3×10⁵ cells per well in 6-well plates. After 24 hours of seeding, the cells were treated with IS at concentrations of 1.25 mM, 5 mM, and 10 mM. Following a 48-hour treatment period, the cells were harvested using trypsin and washed twice with PBS. After the washing steps, the cells were resuspended in 1 ml of fresh media and counted at each concentration using a hematocytometer. From each concentration, 2×10⁵ cells were taken and dissolved in 500 μl of binding buffer, 5 μl of PI, and 5 μl of Annexin V-FITC. The cells were then incubated in the dark at room temperature for 30 minutes. Subsequently, the cells were analyzed for apoptosis using flow cytometry (BD Biosciences, FACS Canto™ Flow Cytometer, Germany) at 488 nm. Untreated cells were used as controls for comparison.

### Cellular Imaging

To assess the morphological changes of the cells following treatment, HT-29 and HCT-116 cells were cultured in 12-well plates at a concentration of 6×10+ cells per well. After 24 hours of seeding, the cells were treated with IS at concentrations of 1.25 mM, 5 mM, and 10 mM. The cells were exposed to IS in triplicate for three different incubation periods: 24 hours, 48 hours, and 72 hours. After the respective incubation periods, the cells were examined using a Nikon microscope equipped with a 20× objective. Images were captured using a digital camera and the NIS-Elements 3.0 software.

### Clonogenic Assay

To assess the colony-forming ability of cells after treatment with IS, a colony forming assay was performed. HCT-116 and HT-29 cells were cultured in 6-well plates at a density of 2×10⁵ cells per well. After 24 hours of seeding, the cells were exposed to IS at concentrations of 1.25 mM, 5 mM, and 10 mM. Following a 48-hour treatment period, the cells were harvested using trypsin and counted for each treatment. From each treatment group, 400, 800, and 1200 cells were plated and incubated for 10 days in 6-well plates. During the incubation period, the media were replenished every alternate day. After 10 days, the colonies were washed with PBS and stained with 0.5% crystalline violet. The cells were then fixed with 6% glutaraldehyde. The colonies were visualized and counted using ImageJ software. The clonogenic efficiency of the treated cells was compared to that of the untreated cells.

### Cell Cycle Assay

The evaluation of the effect of IS on the progression of HCT-116 and HT-29 cell cycle assay involved analysing the G0/G1 phase, Synthesis(S) phase, and G2/M phase. Cells were initially cultured in a 6-well plate with a seeding density of (2×10⁵) cells/well in duplicate. After 24 hours of seeding, the cells were treated with varying concentrations of IS (1.25 mM, 5 mM, and 10 mM). Following a 48-hour processing period, the cells were detached using trypsin and washed twice with refrigerated PBS. The resulting cell pellet was then dissolved in 200 ul of chilled PBS and fixed by adding 800 ul of 70% ethanol dropwise. The samples were stored at −20° C. overnight for later use. After the overnight incubation, the cells were centrifuged at 2000 rpm for 5 minutes at 4° C. The pellet was dissolved in 300 ul of PBS, 50 ul of RNase A (100 ug/ml), and 35 ul of Triton X-100 (0.1%), and incubated at 37° C. for 30 minutes. Subsequently, 20 ul of PI was added from a stock solution of 500 μg/ml, and the samples were incubated in the dark for an additional 30 minutes. Flow cytometry (BD Biosciences, FACS Verse, Germany) was employed to measure PI staining in 1×10³ cells. The Flow Jo software was utilized for the analysis of cell distribution across the different phases of the cell cycle.

### Animal Study

The experimental procedures involving animals were conducted in accordance with the ethical guidelines and regulations set by the Institutional Animal Ethics Committee (IAEC) of the National Institute of Immunology. The IAEC approved these procedures under permit number IAEC #492/18. Additionally, the Institutional Biosafety Committee (IBSC) guidelines were followed to ensure ethical procedures and scientific care during the animal experiments.

Wild-type Balb/c mice were obtained from Jackson Laboratory, USA, and housed in plastic cages with free access to water and food. The mice were randomly assigned to five test groups, which included three control groups: vehicle control, negative control (100 mM sodium propionate), and positive control (AOM (Azoxymethane)/DSS). Additionally, there were two experimental groups receiving 100 mM of indoxyl sulfate, one via intraperitoneal (IP) administration and the other via intrarectal (IR) administration. Each group consisted of six mice. In the AOM/DSS group, AOM was injected intraperitoneally at a dosage of 10 mg/kg bodyweight. The AOM was injected only once on the 1st day of experiment. The positive control mice received 2% DSS for 7 days in the first week, fourth week, and seventh week. Indoxyl sulfate, propionate (negative control), and saline (vehicle control) were administered to the respective groups on a weekly basis. After 12 weeks of indoxyl sulfate administration, the mice were euthanized through cervical dislocation for further analysis.

### Histology

The colon was collected from each mouse for subsequent histology studies. To ensure proper fixation, 10% neutral buffered formalin was used. For histological processing, the tissue samples from the colon and rectum region were initially fixed in 10% saline solution. Subsequently, they were dehydrated using increasing concentrations of ethanol, washed in xylene, and then immersed in molten paraffin wax. The tissue samples were embedded in molten paraffin wax to create a hard block. Using a rotary microtome, sections of the tissue block were cut at a thickness of 4 μm. These sections were transferred to a water bath at 40° C. and then mounted onto glass slides for staining with hematoxylin and eosin. The stained slides were examined under a light microscope at magnifications of 10× and 40×. Images were captured at both magnifications for further analysis. Various parameters, including tumor localization (proximal or distal bowel, rectum, cecum, ileocecal valve), tumor number, cancer stage (dysplasia/adenoma or adenocarcinoma, determined by infiltration into the muscular mucosa), immune cell infiltration, and inflammation, were assessed. Bowel inflammation was evaluated by observing immune cell infiltration and the presence or absence of mucous membranes using a 10× objective. Microscopic examination was performed using an Olympus BX51 microscope.

### Cytokine Study

For the cytokine study, blood samples were collected from the mice. The cytokines of interest, IL-1β, TNF-α, and IL-17A, were analyzed in all groups at three different time points: baseline (time point zero), one month, and two months. The analysis was performed using an ELISA kit from R&D Systems. The experimental procedure for cytokine study followed the instructions provided with the kit. In summary, each well of the ELISA plate was coated with a specific monoclonal antibody against the respective cytokines. Then, 50 ul of standard, control, and sample were added to the appropriate wells. The plate was incubated at room temperature (RT) for 2 hours. After incubation, each well was washed four times with 400 ul of buffer to remove any unbound substances. Subsequently, 100 ul of the secondary antibody conjugate was added to each well and incubated at RT for another 2 hours. Following the second incubation, the wells were washed four times, and 100 ul of substrate solution was added. The plate was then incubated for 30 minutes at RT in the dark. After incubation, 100 ul of stop solution was added to each well to halt the enzymatic reaction. The optical density (OD) of the samples was measured at wavelengths of 450 nm and 570 nm within 30 minutes of adding the stop solution. To obtain the final readings, the absorbance at 570 nm was subtracted from the absorbance at 450 nm.

### Statistical Analysis

In the statistical analysis of the data, one-way ANOVA was employed using SPSS software. The results are shown as mean value (±SEM). P value<0.05 was considered statistically significant, indicating a significant difference in the analyzed variables.

### EXAMPLES

### Example 1

### Effect of IS on Cell Viability

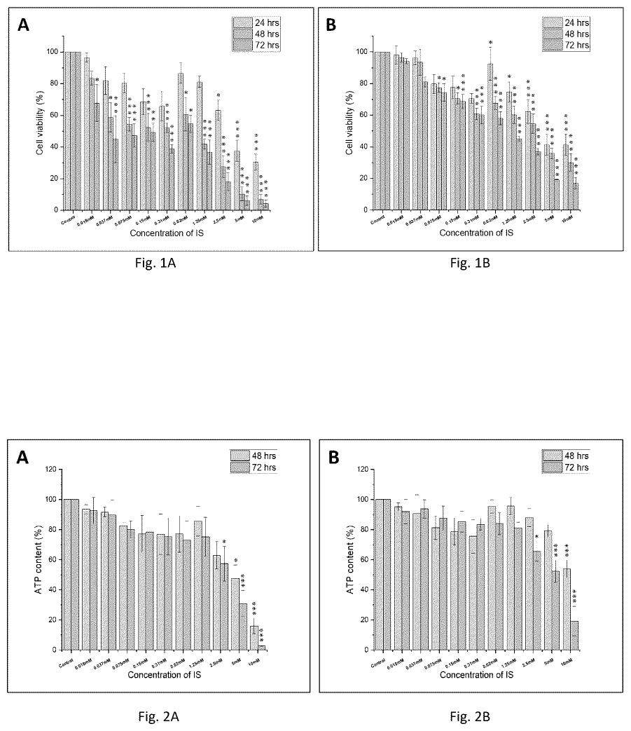

The viability of the cells was assessed using a cell viability assay. Indoxyl sulfate demonstrated a time- and concentration-dependent decrease in cell viability, as illustrated in FIG. 1. Upon exposure to 10 mM of IS, HCT-116 cells exhibited viability of only 30%, 7%, and 4% after 24 hours, 48 hours, and 72 hours, respectively. Similarly, HT-29 cells displayed viability of 41%, 30%, and 17% under the same conditions. The control group, consisting of untreated cells, exhibited 100% viability. Comparison with the control group revealed that indoxyl sulfate exerted a substantial inhibitory effect on the cell viability of adenocarcinoma cells in a time- and concentration-dependent manner. However, a notable observation was made at a specific concentration of 0.62 mM, where cell viability unexpectedly increased. This suggests a potential growth-promoting effect of IS at this particular concentration, reminiscent of the “butyrate paradox” 33. Nonetheless, further investigation is necessary to confirm this finding.

### Example 2

### Effect of IS on ATP Content

The viability of the cells was assessed using a cell viability assay. After 72 hours of treatment, HCT-116 cells exhibited only 3% viability, while HT-29 cells showed 19% viability. The control group, consisting of non-treated cells, maintained 100% viability. These results demonstrate that indoxyl sulfate effectively reduces the viability of HT-29 and HCT-116 cells in a time- and concentration-dependent manner, as depicted in FIG. 2.

As observed in the cell viability assays, the significant change is starting at 1.25 mM IS concentration, hence further assays were carried out at the concentration ranging from 1.25 mM to 10 mM.

### Example 3

### Effect of IS on Membrane Integrity

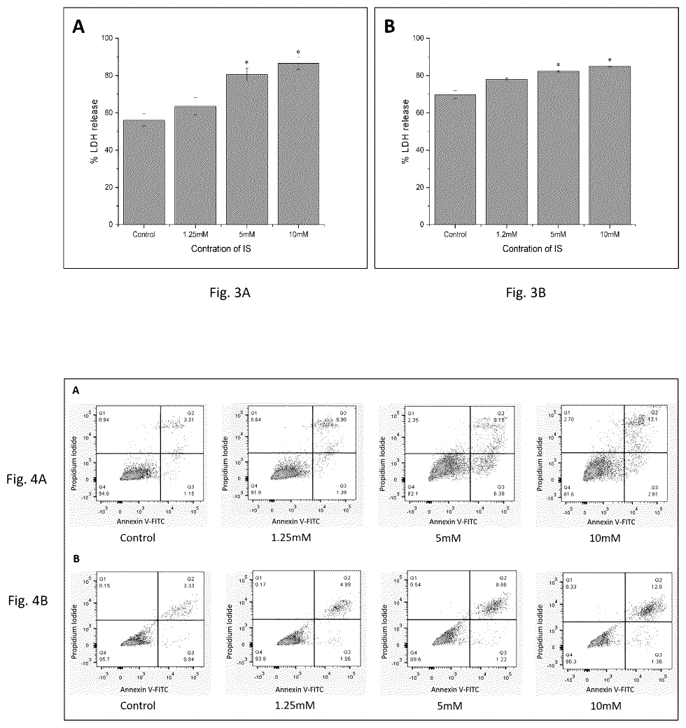

After 48 hours of treatment, the LDH release in the supernatant of HCT-116 cells was 80% and 86% at 5 mM and 10 mM of IS, respectively. Similarly, in HT-29 cells, the LDH release in the supernatant was 82% and 85% at 5 mM and 10 mM of IS, respectively. These results indicate that exposure to IS leads to the loss of membrane integrity in adenocarcinoma cells in a concentration-dependent manner. The highest toxicity was observed at 10 mM, as demonstrated in FIG. 3.

### Example 4

### Effect of IS on Apoptosis

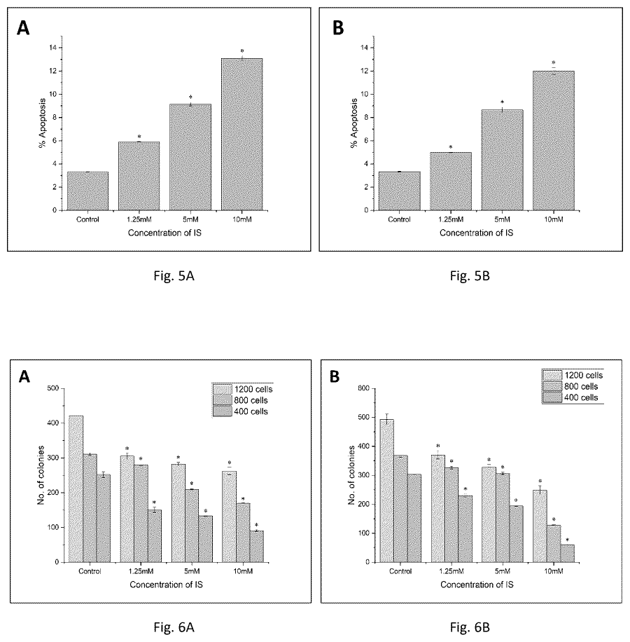

After staining the cells with PI and annexin V, flow cytometry was used to evaluate the effect of IS on cell apoptosis. Annexin V stains cells at both early and late stages of apoptosis, while PI specifically stains cells in late apoptosis and necrosis. Consequently, cells stained with both annexin V and PI were considered in the late stage of apoptosis (right upper quadrant), while cells stained only with annexin V were in the early stage of apoptosis (bottom right quadrant). The flow cytometry dot plot obtained (FIG. 4) clearly demonstrated the apoptotic effect of IS, particularly in the late apoptosis phase (annexin V+, PI+). In the untreated cells, the apoptotic percentage was only 3%. However, after 48 hours of treatment, the apoptotic percentage increased to 9% and 13% in HCT-116 cells at 5 mM and 10 mM concentrations, respectively. Similarly, in HT-29 cells, the apoptotic percentage was 8% and 12% at 5 mM and 10 mM concentrations, respectively (FIG. 5). These results indicate that IS effectively induces apoptosis in cancer cells, with significant apoptotic effects observed at 5 mM and 10 mM concentrations.

### Example 5

### Effect of IS on Cell Morphology

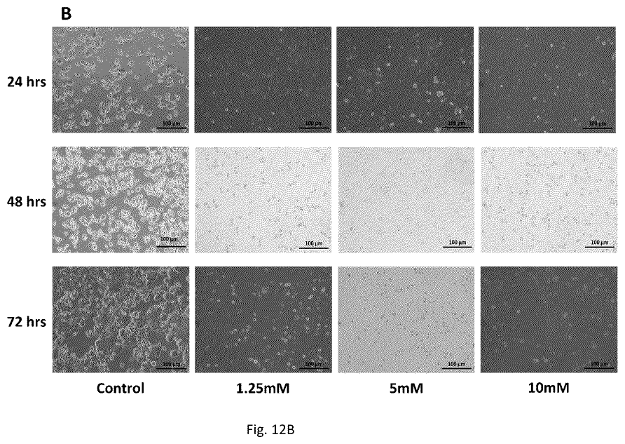

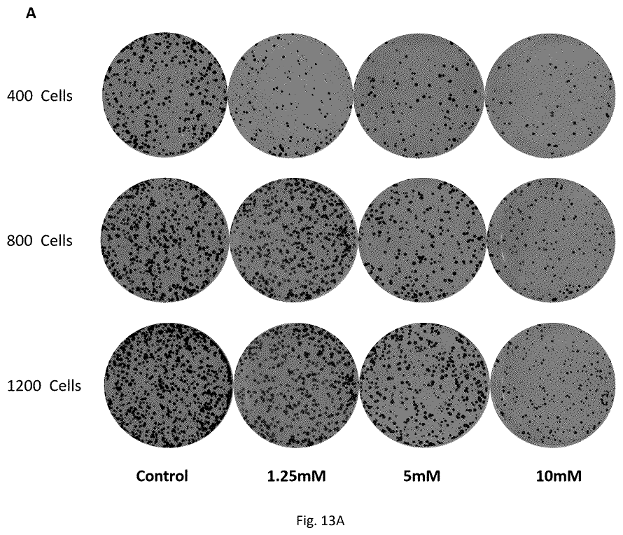

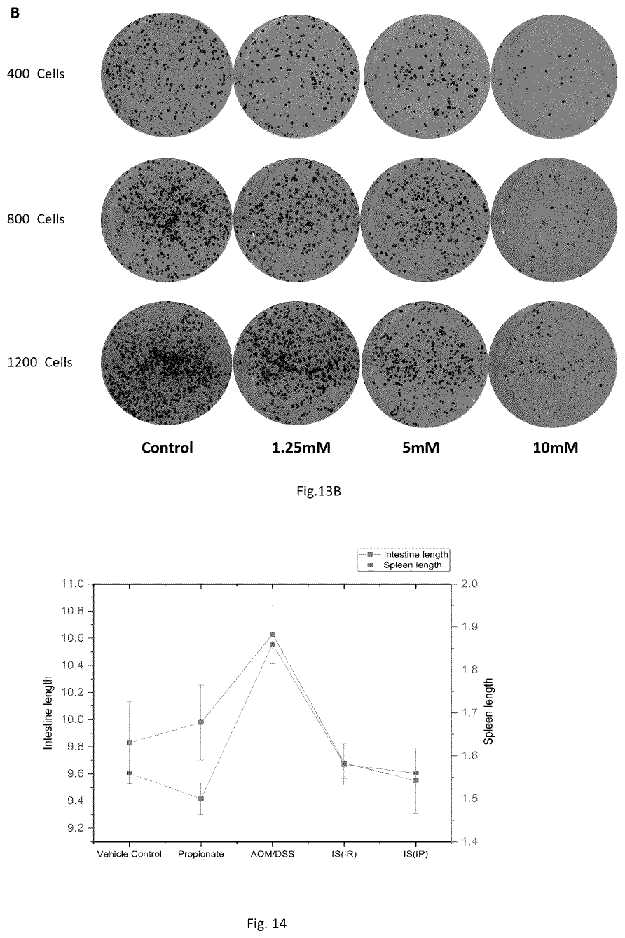

Microscopic observation revealed distinct changes in cell morphology compared to untreated cells (FIGS. 12A-12B). Following IS administration, the cells exhibited a rounded morphology, reduced cell numbers, and detachment from the culture substrate. Moreover, treatment with IS induced the formation of intracellular vesicles. At higher concentrations, the cells were unable to adhere to the culture substrate and instead floated freely in the medium. These findings indicate that IS treatment significantly altered the morphology of HT-29 and HCT-116 cells, leading to rounded cell shape, reduced cell numbers, detachment from the substrate, and the generation of intracellular vesicles. At higher concentrations, the cells exhibited a complete loss of adhesion and floated freely in the medium.

### Example 6

### Effect of IS on Colony Formation Ability of Cancer Cells

The results demonstrated a significant reduction in the number of colonies formed compared to the control (untreated cells), and this reduction occurred in a concentration-dependent manner. Representative images (FIGS. 13A-13B) and quantitative analysis (FIG. 6) of the number of colonies formed by HT-29 and HCT-116 cells after treatment with IS confirmed the cytotoxic effect of IS on adenocarcinoma cells. The images clearly showed a decrease in the number of colonies, indicating the inhibitory impact of IS on the clonogenic potential of the cells. These findings provide evidence that IS exerts a cytotoxic effect on HT-29 and HCT-116 cells by inhibiting colony formation, as demonstrated by both representative images and quantitative analysis.

### Example 7

### Effect of IS on Cell Cycle

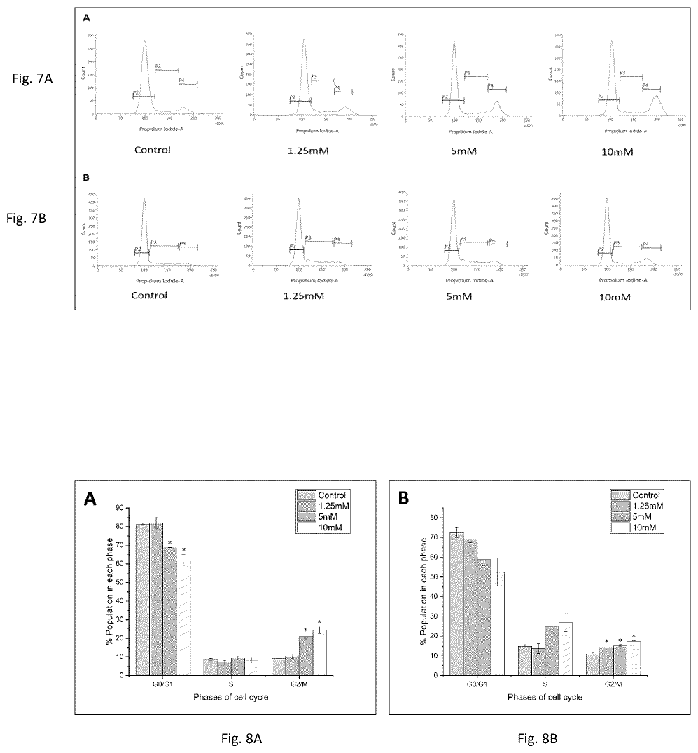

Flow cytometry analysis was performed to evaluate the effect of IS on cell cycle progression. The results revealed a substantial increase in the proportion of cells in the G2/M phase at higher concentrations of IS compared to the proportion of cells in the S-phase. This suggests G2/M cell cycle arrest in response to genotoxic insult at higher doses of IS. Untreated cells served as the negative control. After DNA replication in the S-phase, cells enter the G2 phase to prepare for mitosis. If the G2 phase is halted, cell division is affected, leading to a cessation of cell growth and an inability to progress to the next phase of the cell cycle. Statistical analysis demonstrated a significant increase in the number of cells in the G2/M phase at 5 mM and 10 mM concentrations of IS. In HCT-116 cells, the number of cells in the G2/M phase increased from 18.1±1.99 to 22.7±3.2, while in HT-29 cells, it increased from 14.2±0.5 to 17.3±0.7 (FIG. 7, 8). These findings indicate that IS induces G2/M phase cell cycle arrest in a concentration-dependent manner in HCT-116 and HT-29 cells.

### Example 8

### Animal Study: Effect of IS on Histology

After observing deleterious and anti-cancer effects of indoxyl sulfate colon cancer cells (HCT-116 and HT-29 cells), it was essential to see its inflammatory and damaging effects on normal cells for concretizing the basis that IS is safe of its use as anti-cancer intervention in the colon cancer treatment. We reckoned that studying the effect of IS on normal epithelial colon cells in vivo is more relevant and important than carrying out the same experiment in-vitro using normal cell line. In order to figure out the possibility of using IS in the treatment of colon cancer, in-vivo study is more important, hence we extended our study to in-vivo mode. To assess the potential inflammatory role or deleterious effects of IS on normal colonic cells, an in vivo study was conducted using a balb/c mice model.

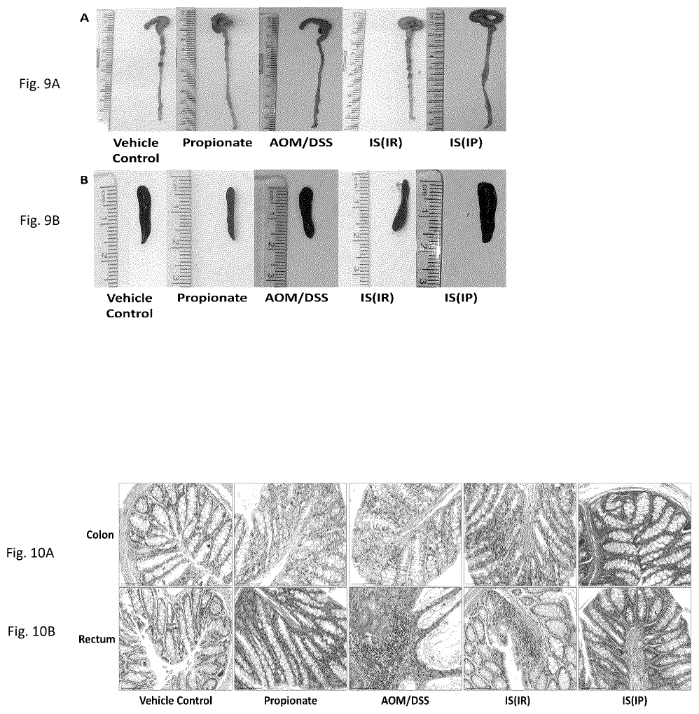

During the animal study, mice were sacrificed at the 12th week, and their body weight was measured. Results showed no significant change in body weight between the IS-treated group and the vehicle and negative control groups. Furthermore, the length of the spleen and intestine was measured using a scale (FIG. 9), and the standard deviations were calculated (Table 1 below). No substantial statistical deviation in the length of the intestine and spleen was observed between the treated, vehicle, and negative control groups (FIG. 13). Histological evaluation of the colon and rectum region was performed using hematoxylin and eosin staining, and images were captured under a light microscope. The histological images from different animal groups (FIG. 10) revealed no noticeable changes in the histology of the colon and rectum region following IS treatment. The morphology of the colon and rectum in the IS-treated group was comparable to that of the vehicle and negative control groups. These results indicate that the administration of indoxyl sulfate did not induce any significant histological damage in mice, suggesting the absence of inflammatory or deleterious effects on normal colonic cells.

TABLE 1
Length of spleen and intestine of different groups

| | Length | Length of |
|---|---|---|
| | of spleen | large intestine |
| Group | (mean ± SD) | (mean ± SD) |
| Vehicle control | 1.56 ± 0.05 | 9.83 ± 0.74 |
| Propionate | 1.5 ± 0.08 | 9.98 ± 0.67 |
| AOM/DSS | 1.86 ± 0.01 | 10.83 ± 0.34 |
| IS (IR) | 1.58 ± 0.11 | 9.61 ± 0.34 |
| IS (IP) | 1.56 ± 0.12 | 9.55 ± 0.58 |

### Example 9

### Effect of IS on Inflammation

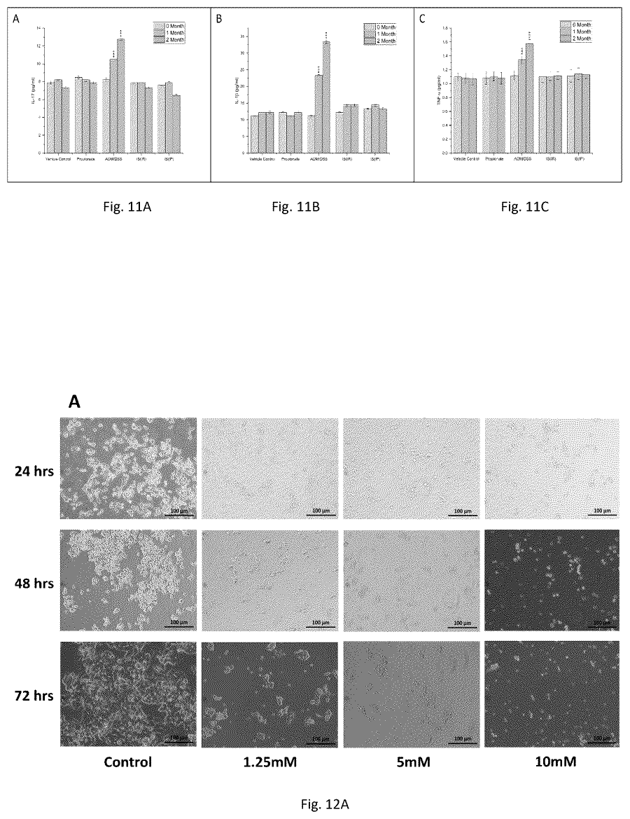

Mice were monitored for symptoms of inflammation, including changes in body weight, stool consistency, and the presence of blood in the stool. During the study, no significant changes were observed in fecal consistency or body weight after IS treatment, indicating the absence of overt inflammatory symptoms. The levels of IL-17A, IL-1β, and TNF-α were measured after IS treatment at different time points and compared to the levels in the vehicle and negative control groups. Statistical analysis revealed no significant differences in the levels of these cytokines between the IS-treated group and the control groups (FIG. 11). Furthermore, the results from the histology study provided further confirmation that IS did not induce harm or inflammation in normal colonic cells of the mice. Taken together, these findings suggest that IS treatment does not result in significant changes in inflammatory cytokine levels and does not induce inflammation or harm normal colonic cells in the mice.

### Analysis and Results

The present invention is about the potential anticancer role of indoxyl sulfate, a gut microbiota-derived metabolite, and its deleterious effects on colon cancer cells. The investigation involved HCT-116 and HT-29 human epithelial adenocarcinoma cells to assess the anticancer potential of indoxyl sulfate. Additionally, an animal study was conducted using balb/c mice to demonstrate the selective deleterious effect of indoxyl sulfate on cancer cells while sparing normal colonic cells.

In the in vitro study, cells were exposed to various doses of indoxyl sulfate ranging from 0.038 mM to 10 mM for three different incubation periods (24 hours, 48 hours, and 72 hours). A decrease in cell proliferation efficiency was observed after 48 hours of treatment, with the most significant effects observed at concentrations of 5 mM and 10 mM of indoxyl sulfate. All tested subjects exhibited a significant decrease in cell viability and cellular ATP content in a time- and dose-dependent manner. Notably, a concentration of 10 mM of indoxyl sulfate at 72 hours proved to be the most cytotoxic to cancer cells. However, an intriguing finding emerged at a specific concentration of 0.62 mM, where cell viability exhibited a sudden increase. This suggests that indoxyl sulfate may exert a growth-promoting effect at this particular concentration, similar to the phenomenon known as the “butyrate paradox”, but further investigation is required to confirm this observation. In a research report from 2018, it was demonstrated that butyrate acts as a histone deacetylase inhibitor (HDACi) only in normal cells at a specific concentration, namely 0.5 mM. Above this concentration, it does not function as an HDACi in normal cells. Decrease in cellular ATP content indicates the diminished cellular metabolic activity and may be linked with cell death. Considering these findings. the apoptotic effect of indoxyl sulfate was examined. After a 48-hour treatment. cells displayed an increase in apoptosis in a concentration-dependent manner, with 10 mM IS being the most cytotoxic, resulting in a 10% increase in cell apoptosis compared to the control. A study conducted in 2018 by R. J. Ellis et al., demonstrated that IS induces apoptosis in human kidney proximal tubular epithelial cells (PTECs). Another study on the osteoblast cell line MC3T3-E1 also confirmed that IS acts as a bone toxin by inducing cell apoptosis and inhibiting differentiation. Evaluation of colony-forming ability at various concentrations following a 48-hour IS treatment revealed that IS inhibits the colony-forming ability of cells in a concentration-dependent manner. Additionally. cell morphology was examined using a light microscope after the 48-hour treatment, which revealed a reduced number of cells. distorted cell morphology. formation of intracellular vesicles, and cells floating in the media. The most cytotoxic effects were observed at 5 mM and 10 mM concentrations. Cells showed a significant increase in lactate dehydrogenase enzyme levels at 5 mM and 10 mM after 48 hours of treatment. indicating loss of membrane integrity. Following the 48-hour treatment. cells displayed a higher distribution in the G2/M phase compared to the control, indicating that IS causes G2/M cell cycle arrest in a concentration-dependent manner, with the most cytotoxic effects observed at 10 mM. The effect of IS on the rat colonic cell line IEC-6 was examined in 2019 by Adesso et al., revealing that indoxyl sulfate induces the release of reactive oxygen species (ROS). leading to increased oxidative cell injury and affecting associated antioxidant enzymes. To account for this study. cells were also assessed for oxidative stress in this invention by staining them with dithioethidium bromide (DHE) after 48 hours of treatment. However, indoxyl sulfate did not cause any production of ROS in human adenocarcinoma cells. The potential for IS to cause DNA damage was also investigated. Cells lysate was taken after IS treatment and comet assay was done but it does not show formation of any comet.

The various cell assays conducted in in this invention demonstrated the deleterious effect of indoxyl sulfate on HCT-116 and HT-29 human epithelial adenocarcinoma cells. However. an important question arises regarding whether IS causes any damage to normal cells. To address this, an in vivo study was performed using a balb/c mice model. The results of the in vivo study revealed that indoxyl sulfate does not cause any histological damage to the normal colonic cells of mice. This finding provides evidence that IS does not have a detrimental effect on normal cells. Sugimura et al., also showed that an indole derivate, indole-3-cabinol (I3C) can promote the apoptosis in HCT-116 and LoVo cells without harming the normal colon epithelial cells. Additionally, the levels of three different inflammatory cytokines (IL-17A, IL-1β, and TNF-α) were examined before and after treatment with indoxyl sulfate. The results confirmed that IS does not induce any inflammation. In previous reports, other indole derivatives such as indole-3-propionic acid (IPA) and indole-3-acetic acid (IAA) were reported to have an indirect anti-inflammatory impact by manipulating myeloperoxidase (MPO). Considering all the results obtained from both the in vivo and in vitro studies, it can be concluded that indoxyl sulfate exhibits an anticancer effect and is deleterious to colon cancer cells specifically. Importantly, it does not cause any harm or inflammation to normal colonic cells.

The effect of indoxyl sulfate on tumor advancement is likely related to the reversal of epithelial-mesenchymal transition (EMT), as observed in studies on other metabolite. EMT reversal or inhibition has been shown to slow down cell movement. diapedesis (the migration of cells through blood vessel walls), and the formation of metastases, as reported by Sari et al. In addition to the decrease in EMT, they also observed a decrease in the expression of mesenchymal markers and a simultaneous increase in the expression of epithelial markers (ZO-1 and E-cadherin). The suppression of EMT can be a key factor in inhibiting cell movement and diapedesis.

While indoxyl sulfate is a well-known uremic toxin that exhibits cytotoxic effects in the case of chronic kidney diseases, its effects on colon cancer cells have been less studied. Other indole derivative indole-3-cabinol (I3C) has been well studied using HT-29, HCT-116 and DLDI cell lines and found decrease in cell viability and accelerated apoptosis as an AHR agonist. Another study showed that I3C can suppress the growth and migration of human colorectal carcinoma LoVo cells. Therefore, this invention is aimed to understand the anticancer and selective deleterious role of indoxyl sulfate on colon epithelial adenocarcinoma cells and normal colonic cells in mice. Results demonstrated that IS induces apoptosis, cell cycle arrest, loss of membrane integrity, disturbed cell morphology, inhibition of cell viability, and inhibition of colony formation in adenocarcinoma cells. In contrast, IS did not cause any harm or inflammation in normal colonic cells of balb/c mice. Based on the results obtained from different assays and the animal study, it can be concluded that indoxyl sulfate possesses anticancer potential against colon cancer cells and possibly other types of cancer cells as well. Furthermore, since indoxyl sulfate does not harm or inflame normal colonic cells, it can be considered safe for use as an anticancer agent and may have implications in future applications for colon cancer treatment.

### REFERENCES

(1) Morgan, E.: Arnold, M.: Gini, A.: Lorenzoni, V.: Cabasag, C. J.: Laversanne, M.: Vignat, J.: Ferlay, J.: Murphy, N.: Bray, F. Global Burden of Colorectal Cancer in 2020 and 2040: Incidence and Mortality Estimates from GLOBOCAN. Gut 2022, gutjnl-2022-327736. https://doi.org/10.1136/gutjnl-2022-327736.
(2) Jaye, K.: Li, C. G.: Chang, D.: Bhuyan, D. J. The Role of Key Gut Microbial Metabolites in the Development and Treatment of Cancer. Gut Microbes 2022, 14 (1), 2038865. https://doi.org/10.1080/19490976.2022.2038865.
(3) Zhao, L.-Y.: Mei, J.-X.: Yu, G.: Lei, L.: Zhang, W.-H.: Liu, K.: Chen, X.-L.: Kolat, D.: Yang, K.: Hu, J.-K. Role of the Gut Microbiota in Anticancer Therapy: From Molecular Mechanisms to Clinical Applications. Sig Transduct Target Ther 2023, 8 (1), 1-27. https://doi.org/10.1038/s41392-023-01406-7.
(4) Kovtonyuk, L. V.: McCoy, K. D. Microbial Metabolites and Immunotherapy: Basic Rationale and Clinical Indications. Semin Immunol 2023, 67, 101755. https://doi.org/10.1016/j.smim.2023.101755.
(5) Zhao, H.: Wang, D.: Zhang, Z.: Xian, J.: Bai, X. Effect of Gut Microbiota-Derived Metabolites on Immune Checkpoint Inhibitor Therapy: Enemy or Friend? Molecules 2022, 27 (15), 4799. https://doi.org/10.3390/molecules27154799.
(6) Li, X.: Zhang, S.: Guo, G.: Han, J.: Yu, J. Gut Microbiome in Modulating Immune Checkpoint Inhibitors. EBioMedicine 2022, 82, 104163. https://doi.org/10.1016/j.ebiom.2022.104163.
(7) Lu, Y.: Yuan, X.: Wang, M.: He, Z.: Li, H.: Wang, J.: Li, Q. Gut Microbiota Influence Immunotherapy Responses: Mechanisms and Therapeutic Strategies. J Hematol Oncol 2022, 15 (1), 47. https://doi.org/10.1186/s13045-022-01273-9.
(8) Beaumont, M.: Andriamihaja, M.: Armand, L.: Grauso, M.: Jaffrézic, F.: Laloë, D.: Moroldo, M.: Davila, A.-M.; Tome, D.: Blachier, F.: Lan, A. Epithelial Response to a High-Protein Diet in Rat Colon. BMC Genomics 2017, 18 (1), 116. https://doi.org/10.1186/s12864-017-3514-z.
(9) Tennoune, N.: Andriamihaja, M.: Blachier, F. Production of Indole and Indole-Related Compounds by the Intestinal Microbiota and Consequences for the Host: The Good, the Bad, and the Ugly. Microorganisms 2022, 10 (5), 930. https://doi.org/10.3390/microorganisms10050930.
(10) Singh, A. K.: Raj, V.: Saha, S. Indole-Fused Azepines and Analogues as Anticancer Lead Molecules: Privileged Findings and Future Directions. European Journal of Medicinal Chemistry 2017, 142, 244-265. https://doi.org/10.1016/j.ejmech.2017.07.042.
(11) Dixit, A.: Pathak, D.: Sharma, G. A REVIEW ON DOCKING STUDIES OF INDOLE MOIETY AS POTENT INHIBITOR OF TUBULIN POLYMERIZATION. European Chemical Bulletin 2017.
(12) Dhiman, A.: Sharma, R.: Singh, R. K. Target-Based Anticancer Indole Derivatives and Insight into Structure-activity Relationship: A Mechanistic Review Update (2018-2021). Acta Pharm Sin B 2022, 12 (7), 3006-3027. https://doi.org/10.1016/j.apsb.2022.03.021.
(13) Kaur, B.: Venugopal, S.: Verma, A.: Sahu, S. K.: Wadhwa, P.: Kumar, D.: Sharma, A. Recent Developments in the Synthesis and Anticancer Activity of Indole and Its Derivatives. Curr Org Synth 2023, 20 (4), 376-394. https://doi.org/10.2174/1570179419666220509215722.
(14) Wan, Y.: Li, Y.: Yan, C.: Yan, M.: Tang, Z. Indole: A Privileged Scaffold for the Design of Anti-Cancer Agents. Eur J Med Chem 2019, 183, 111691. https://doi.org/10.1016/j.ejmech.2019.111691.
(15) Dadashpour, S.: Emami, S. Indole in the Target-Based Design of Anticancer Agents: A Versatile Scaffold with Diverse Mechanisms. Eur J Med Chem 2018, 150, 9-29. https://doi.org/10.1016/j.ejmech.2018.02.065.
(16) Chadha, N.: Silakari, O. Indoles as Therapeutics of Interest in Medicinal Chemistry: Bird's Eye View. Eur J Med Chem 2017, 134, 159-184. https://doi.org/10.1016/j.ejmech.2017.04.003.
(17) Skye, S. M.: Hazen, S. L. Microbial Modulation of a Uremic Toxin. Cell Host Microbe 2016, 20 (6), 691-692. https://doi.org/10.1016/j.chom.2016.11.005.
(18) Liu, W.-C.: Tomino, Y.: Lu, K.-C. Impacts of Indoxyl Sulfate and P-Cresol Sulfate on Chronic Kidney Disease and Mitigating Effects of AST-120. Toxins (Basel) 2018, 10 (9). https://doi.org/10.3390/toxins10090367.
(19) Sári, Z.: Mikó, E.: Kovács, T.: Boratkó, A.: Ujlaki, G.: Jankó, L.: Kiss, B.: Uray, K.: Bai, P. Indoxylsulfate, a Metabolite of the Microbiome, Has Cytostatic Effects in Breast Cancer via Activation of AHR and PXR Receptors and Induction of Oxidative Stress. Cancers 2020, 12 (10), 2915. https://doi.org/10.3390/cancers12102915.
(20) Roager, H. M.: Licht, T. R. Microbial Tryptophan Catabolites in Health and Disease. Nat Commun 2018, 9 (1), 3294. https://doi.org/10.1038/s41467-018-05470-4.
(21) Sári, Z.: Mikó, E.: Kovács, T.: Jankó, L.: Csonka, T.: Lente, G.: Sebö, É.: Tóth, J.: Tóth, D.: Árkosy, P.: Boratkó, A.: Ujlaki, G.: Török, M.: Kovács, I.: Szabó, J.: Kiss, B.: Méhes, G.: Goedert, J. J.: Bai, P. Indolepropionic Acid, a Metabolite of the Microbiome, Has Cytostatic Properties in Breast Cancer by Activating AHR and PXR Receptors and Inducing Oxidative Stress. Cancers 2020, 12 (9), 2411. https://doi.org/10.3390/cancers 12092411.
(22) Kovács, P.: Csonka, T.: Kovács, T.: Sári, Z.: Ujlaki, G.: Sipos, A.: Karányi, Z.: Szeöcs, D.: Hegedüs, C.: Uray, K.: Jankó, L.: Kiss, M.: Kiss, B.: Laoui, D.: Virág, L.: Méhes, G.: Bai, P.; Miko, E. Lithocholic Acid, a Metabolite of the Microbiome, Increases Oxidative Stress in Breast Cancer. Cancers (Basel) 2019, 11 (9), 1255. https://doi.org/10.3390/cancers11091255.
(23) Mikó, E.: Vida, A.: Kovács, T.: Ujlaki, G.: Trencsényi, G.: Márton, J.: Sári, Z.: Kovács, P.: Boratkó, A.: Hujber, Z.: Csonka, T.: Antal-Szalmás, P.: Watanabe, M.: Gombos, I.: Csoka, B.: Kiss, B.: Vígh, L.: Szabó, J.: Méhes, G.: Sebestyén, A.: Goedert, J. J.: Bai, P. Lithocholic Acid, a Bacterial Metabolite Reduces Breast Cancer Cell Proliferation and Aggressiveness. Biochim Biophys Acta Bioenerg 2018, 1859 (9), 958-974. https://doi.org/10.1016/j.bbabio.2018.04.002.
(24) Kovács, T.: Mikó, E.: Vida, A.: Sebö, É.: Toth, J.: Csonka, T.: Boratkó, A.: Ujlaki, G.: Lente, G.: Kovács, P.: Tóth, D.: Árkosy, P.: Kiss, B.: Méhes, G.: Goedert, J. J.: Bai, P. Cadaverine, a Metabolite of the Microbiome, Reduces Breast Cancer Aggressiveness through Trace Amino Acid Receptors. Sci Rep 2019, 9 (1), 1300. https://doi.org/10.1038/s41598-018-37664-7.
(25) Thirunavukkarasan, M.: Wang, C.: Rao, A.: Hind, T.: Teo, Y. R.; Siddiquee, A. A.-M.: Goghari, M. A. I.: Kumar, A. P.: Herr, D. R. Short-Chain Fatty Acid Receptors Inhibit Invasive Phenotypes in Breast Cancer Cells. PLOS ONE 2017, 12 (10), e0186334. https://doi.org/10.1371/journal.pone.0186334.
(26) Tang. W.: Putluri, V.: Ambati, C. R.: Dorsey, T. H.: Putluri, N.: Ambs, S. Liver- and Microbiome-Derived Bile Acids Accumulate in Human Breast Tumors and Inhibit Growth and Improve Patient Survival. Clin Cancer Res 2019, 25 (19), 5972-5983. https://doi.org/10.1158/1078-0432.CCR-19-0094.
(27) Sári, Z.: Kovács, T.: Csonka, T.: Török, M.: Sebö, É.: Toth, J.: Tóth, D.: Mikó, E.: Kiss, B.: Szeöcs, D.: Uray, K.: Karányi, Z.: Kovács, I.: Méhes, G.: Árkosy, P.: Bai, P. Fecal Expression of Escherichia Coli Lysine Decarboxylase (LdcC) Is Downregulated in E-Cadherin Negative Lobular Breast Carcinoma. Physiol Int 2020, 107 (2), 349-358. https://doi.org/10.1556/2060.2020.00016.
(28) Salimi, V.: Shahsavari, Z.: Safizadeh, B.: Hosseini, A.: Khademian, N.: Tavakoli-Yaraki, M. Sodium Butyrate Promotes Apoptosis in Breast Cancer Cells through Reactive Oxygen Species (ROS) Formation and Mitochondrial Impairment. Lipids Health Dis 2017, 16 (1), 208. https://doi.org/10.1186/s12944-017-0593-4.
(29) Goldberg, A. A.: Beach, A.: Davies, G. F.: Harkness, T. A. A.: LeBlanc, A.: Titorenko, V. I. Lithocholic Bile Acid Selectively Kills Neuroblastoma Cells, While Sparing Normal Neuronal Cells. Oncotarget 2011, 2 (10), 761-782.
(30) Sreekanth, V.: Bansal, S.: Motiani, R. K.: Kundu, S.: Muppu, S. K.: Majumdar, T. D.: Panjamurthy, K.: Sengupta, S.: Bajaj, A. Design, Synthesis, and Mechanistic Investigations of Bile Acid-Tamoxifen Conjugates for Breast Cancer Therapy. Bioconjug Chem 2013, 24 (9), 1468-1484. https://doi.org/10.1021/bc300664k.
(31) Gafar, A. A.: Draz, H. M.: Goldberg, A. A.: Bashandy, M. A.: Bakry, S.: Khalifa, M. A.: Abu hair, W.: Titorenko, V. I.: Sanderson, J. T. Lithocholic Acid Induces Endoplasmic Reticulum Stress, Autophagy and Mitochondrial Dysfunction in Human Prostate Cancer Cells. PeerJ 2016, 4, e2445. https://doi.org/10.7717/peerj.2445.
(32) Luu, T. H.: Bard, J.-M.: Carbonnelle, D.: Chaillou, C.: Huvelin, J.-M.: Bobin-Dubigeon, C.: Nazih, H. Lithocholic Bile Acid Inhibits Lipogenesis and Induces Apoptosis in Breast Cancer Cells. Cell Oncol (Dordr) 2018, 41 (1), 13-24. https://doi.org/10.1007/s13402-017-0353-5.
(33) Ryu, S. H.: Kaiko, G. E.: Stappenbeck, T. S. Cellular Differentiation: Potential Insight into Butyrate Paradox? Molecular & Cellular Oncology 2018, 5 (3). https://doi.org/10.1080/23723556.2016.1212685.
(34) Burgess, D. J. Warburg behind the Butyrate Paradox? Nat Rev Cancer 2012, 12 (12), 798-799. https://doi.org/10.1038/nrc3401.
(35) Gibson, P. R.: Rosella, O.: Wilson, A. J.: Mariadason, J. M.: Rickard, K.: Byron, K.: Barkla, D. H. Colonic Epithelial Cell Activation and the Paradoxical Effects of Butyrate. Carcinogenesis 1999, 20 (4), 539-544. https://doi.org/10.1093/carcin/20.4.539.
(36) Braissant, O.: Astasov-Frauenhoffer, M.: Waltimo, T.: Bonkat, G. A Review of Methods to Determine Viability, Vitality, and Metabolic Rates in Microbiology. Front Microbiol 2020, 11, 547458. https://doi.org/10.3389/fmicb.2020.547458.
(37) Ellis, R. J.: Small, D. M.: Ng, K. L.: Vesey, D. A.: Vitetta, L.: Francis, R. S.: Gobe, G. C.: Morais, C. Indoxyl Sulfate Induces Apoptosis and Hypertrophy in Human Kidney Proximal Tubular Cells. Toxicol Pathol 2018, 46 (4), 449-459. https://doi.org/10.1177/0192623318768171.
(38) Kim, Y.-H.: Kwak, K.-A.: Gil, H.-W.: Song, H.-Y.: Hong, S.-Y. Indoxyl Sulfate Promotes Apoptosis in Cultured Osteoblast Cells. BMC Pharmacol Toxicol 2013, 14, 60. https://doi.org/10.1186/2050-6511-14-60.
(39) Pan, Z.: Zhang, X.: Yu, P.: Chen, X.: Lu, P.: Li, M.: Liu, X.: Li, Z.: Wei, F.: Wang, K.: Zheng, Q.: Li, D. Cinobufagin Induces Cell Cycle Arrest at the G2/M Phase and Promotes Apoptosis in Malignant Melanoma Cells. Frontiers in Oncology 2019, 9.
(40) Qin, F.: Wang, C. Y.: Wang, C.-G.: Chen, Y.: Li, J.-J.: Li, M.-S.: Zhu, Y.-K.: Lee, S. K.: Wang, H.-S. Undescribed Isoquinolines from Zanthoxylum Nitidum and Their Antiproliferative Effects against Human Cancer Cell Lines. Phytochemistry 2023, 205, 113476. https://doi.org/10.1016/j.phytochem.2022.113476.
(41) Adesso, S.: Ruocco, M.: Rapa, S. F.: Dal Piaz, F.: Di Iorio, B. R.: Popolo, A.: Autore, G.: Nishijima, F.: Pinto, A.: Marzocco, S. Effect of Indoxyl Sulfate on the Repair and Intactness of Intestinal Epithelial Cells: Role of Reactive Oxygen Species' Release. Int J Mol Sci 2019, 20 (9). https://doi.org/10.3390/ijms20092280.
(42) Sugimura, N.: Li, Q.: Chu, E. S. H.: Lau, H. C. H.: Fong, W.: Liu, W.: Liang, C.: Nakatsu, G.: Su, A. C. Y.: Coker, O. O.: Wu, W. K. K.: Chan, F. K. L.: Yu, J. Lactobacillus Gallinarum Modulates the Gut Microbiota and Produces Anti-Cancer Metabolites to Protect against Colorectal Tumourigenesis. Gut 2021, 71 (10), 2011-2021. https://doi.org/10.1136/gutjnl-2020-323951.
(43) Alexeev, E. E.: Dowdell, A. S.: Henen, M. A.; Lanis, J. M.: Lee, J. S.: Cartwright, I. M.: Schaefer, R. E. M.: Ornelas, A.: Onyiah, J. C.: Vögeli, B.: Colgan, S. P. Microbial-Derived Indoles Inhibit Neutrophil Myeloperoxidase to Diminish Bystander Tissue Damage. FASEB J 2021, 35 (5), e21552. https://doi.org/10.1096/fj.202100027R.
(44) Vergara, D.: Simeone, P.: Damato, M.: Maffia, M.: Lanuti, P.: Trerotola, M. The Cancer Microbiota: EMT and Inflammation as Shared Molecular Mechanisms Associated with Plasticity and Progression. J Oncol 2019, 2019, 1253727. https://doi.org/10.1155/2019/1253727.
(45) Patel, M.; McAllister, M.; Nagaraju, R.; Badran, S. S. F. A.; Edwards, J.; McBain, A. J.; Barriuso, J.; Aziz, O. The Intestinal Microbiota in Colorectal Cancer Metastasis-Passive Observer or Key Player? Crit Rev Oncol Hematol 2022, 180, 103856. https://doi.org/10.1016/j.critrevonc.2022.103856.
(46) Kim, H. Y.; Yoo, T.-H.; Hwang, Y.; Lee, G. H.; Kim, B.; Jang, J.; Yu, H. T.: Kim, M. C.; Cho, J.-Y.; Lee, C. J.; Kim, H. C.; Park, S.; Lee, W.-W. Indoxyl Sulfate (IS)-Mediated Immune Dysfunction Provokes Endothelial Damage in Patients with End-Stage Renal Disease (ESRD). Sci Rep 2017, 7 (1), 3057. https://doi.org/10.1038/s41598-017-03130-z.
(47) Ragi, N.; Pallerla, P.; Babi Reddy Gari, A. R.; Lingampelly, S. S.; Ketavarapu, V.; Addipilli, R.; Chirra, N.; Kantevari, S.; Yadla, M.; Sripadi, P. Assessment of Uremic Toxins in Advanced Chronic Kidney Disease Patients on Maintenance Hemodialysis by LC-ESI-MS/MS. Metabolomics 2023, 19 (3), 14. https://doi.org/10.1007/s11306-023-01978-z.
(48) Megna, B. W.; Carney, P. R.; Nukaya, M.; Geiger, P.; Kennedy, G. D. Indole-3-Carbinol Induces Tumor Cell Death: Function Follows Form. J Surg Res 2016, 204 (1), 47-54. https://doi.org/10.1016/j.jss.2016.04.021.
(49) Lee, J. Y.; Lim, H. M.; Lee, C. M.; Park, S.-H.; Nam, M. J. Indole-3-Carbinol Inhibits the Proliferation of Colorectal Carcinoma LoVo Cells through Activation of the Apoptotic Signaling Pathway. Hum Exp Toxicol 2021, 40 (12), 2099-2112. https://doi.org/10.1177/09603271211021475.

We claim:

1. An anticancer agent comprising Indoxyl Sulfate for treatment of colorectal cancer.

2. The anticancer agent of claim 1, wherein the Indoxyl Sulfate is present at a concentration range of 0.038 mM to 10 mM.

3. The anticancer agent of claim 1, wherein the Indoxyl Sulfate is present at a concentration of 10 mM.

4. The anticancer agent of claim 1, wherein the Indoxyl Sulfate is present at a concentration of 5 mM.

5. A pharmaceutical composition for inhibiting activity of colon cancer cells comprising:
a therapeutically effective amount of the anticancer agent of claim 1; and
a pharmaceutically acceptable excipient.

6. A method for treating or preventing colorectal cancer in a subject comprising:
providing a therapeutically effective amount of Indoxyl Sulfate; and
administering, to the subject, the therapeutically effective amount of Indoxyl Sulfate and/or any mixture containing the Indoxyl Sulfate.

7. The method of claim 6, wherein the Indoxyl Sulfate is administered alone in a pharmaceutical composition or the Indoxyl Sulfate is administered in combination with another cancer therapy.

8. The method of claim 7, wherein the other cancer therapy consists of any one or more of surgery, chemotherapy, immunotherapy or radiation therapy.

9. The method of claim 6, wherein the colorectal cancer is a locally advanced cancer or a metastatic cancer.

10. The method of claim 6, wherein a diagnosis of the subject is Stage 0 cancer, Stage I cancer, Stage II cancer, Stage III cancer or Stage IV cancer.

11. The method of claim 6, wherein the Indoxyl Sulfate is administered in any one or more pharmaceutical preparation, a nutritional preparation or a food preparation.

12. The method of claim 6, wherein the Indoxyl Sulfate is administered in a dosage amount ranging from 100 mg/Kg body weight to 125 mg/Kg body weight of the subject under treatment.

13. The method of claim 6, wherein the subject is a mammal.

14. A method of using Indoxyl Sulfate as an anticancer agent for treatment of colorectal cancer in a subject.
